(19) United States
(12) Patent Application Publication
(10) Pub. No.: US 2009/0298116 A1
(43) Pub. Date: Dec. 3, 2009

(54) CELL CULTURE APPARATUS HAVING DIFFERENT MICRO-WELL TOPOGRAPHY

(76) Inventors: Ye Fang, Painted Post, NY (US); Jianguo Wang, Horseheads, NY (US); Ying Zhang, Corning, NY (US)

Correspondence Address: CORNING INCORPORATED, SP-TI-3-1, CORNING, NY, 14831, US

(21) Appl. No.: 12/471,835

(22) Filed: May 26, 2009

Related U.S. Application Data
(60) Provisional application No. 61/130,369, filed on May 30, 2008.

(51) Int. Cl. C12Q 1/02; C12M 1/00; G03F 7/20

(52) U.S. Cl. 435/29; 435/305.2; 430/320

(57) ABSTRACT

A cell culture apparatus includes a substrate having formed therein a micro-well array, the micro-well array comprising a plurality of micro-wells. Each micro-well is defined by a curved surface which is concave.

### CROSS REFERENCE TO RELATED APPLICATION

This Application claims the benefit of U.S. Provisional Application Ser. No. 61/130,369 filed May 30, 2008 and entitled “Cell Culture Apparatus Having Different Micro-well Topography”.

### FIELD

The invention relates generally to apparatus for cultivating biological cells. More specifically, the invention relates to an apparatus for cultivating three-dimensional multicellular clusters of cells.

### BACKGROUND

Traditionally, in-vitro models for biomedical studies are based on two-dimensional cell cultures, where cells are cultured on a flat surface. However, despite the experimental convenience and good cell viabilities of two-dimensional cell cultures, it has been commonly acknowledged that cell behaviors, such as phenotypes, function, and regulation of signaling pathways, can be fundamentally different between a two-dimensional cell layer and a complex three-dimensional multicellular cluster. A good example is a cancer study where researchers found that three-dimensional malignant breast tumor cells can revert back to their original states with addition of a certain antibody—this curing effect had never been observed in two-dimensional culture. (Bin Kim J., Stein R. and O'Hare M. J., “Three-dimensional in vitro tissue culture models of breast cancer—a review,” Breast Cancer Research and Treatment 2004, 85(3):281-291.) Because the three-dimensional culture systems share more similarities with the physiological environments found in the living organism, the development of a more effective in-vivo three-dimensional cell culture system would be critical for many research areas, such as new drug development, stem and cancer research, and tissue engineering.

The essential environment of in-vivo cells is the existence of extracellular matrix, which provides cell-substrate interaction support and facilitates nutrition and metabolic waste transportation. Research on cell-substrate interactions have shown that for a specific substrate surface, different cells can show significantly different behaviors in terms of, for example, cell adhesion, morphology, orientation, mobility, and bioactivities. The ultimate aim of cell-substrate interaction research is thus to obtain the optimum substrate surface for directing cell culture and development. However, much of the present research dwells on obtaining three-dimensional cell clusters at defined locations by physically or chemically confining cell growth. In physical confinement, an array of wells is fabricated. Subsequently, cells in culture are confined within each well to form multicellular clusters of cells. Typically, the wells have steep sidewalls and a flat bottom so that even with confinement the cells grow on a flat surface. Thus, the cells do not interact with a true three-dimensional surface.

### SUMMARY

In one aspect, the invention relates to a cell culture apparatus which comprises a substrate having formed therein a micro-well array. The micro-well array comprises a plurality of micro-wells. Each micro-well is defined by a curved surface which is concave.

In another aspect, the invention relates to a method of making a cell culture apparatus which comprises forming a micro-dot array on a first substrate, where the micro-dot array comprises a plurality of micro-dots and each micro-dot is a negative copy of a micro-well having a curved surface which is concave. The method further includes impressing the micro-dot array into a second substrate to form a micro-well array in the second substrate, where the micro-well array comprises a plurality of micro-wells, each of which has a curved surface which is concave.

Other features and advantages of the invention will be apparent from the following description and the appended claims.

### BRIEF DESCRIPTION OF DRAWINGS

The accompanying drawings, described below, illustrate typical embodiments of the invention and are not to be considered limiting of the scope of the invention, for the invention may admit to other equally effective embodiments. The figures are not necessarily to scale, and certain features and certain views of the figures may be shown exaggerated in scale or in schematic in the interest of clarity and conciseness.

FIG. 1 is a perspective view of a cell culture apparatus.

FIG. 2 is a cross-sectional view of a cell culture apparatus depicting various micro-well geometries.

FIG. 3 is a perspective view of a cell culture apparatus depicting micro-channels between micro-wells.

FIG. 4 is a cross-sectional view of a cell culture apparatus with a recessed micro-well array and illustrating micro-well parameters.

FIG. 5 is a top view of a cell culture apparatus having a plurality of micro-well arrays.

FIG. 6 is a top view of a cell culture apparatus having a plurality of recessed micro-well arrays.

FIG. 7A illustrates photo-patterning of a substrate.

FIG. 7B shows micro-posts formed on the substrate of FIG. 7A by etching.

FIG. 7C shows the micro-posts of FIG. 7B shaped into micro-dots.

FIG. 7D shows a first substrate including micro-dots brought into contact with a second substrate.

FIG. 7E shows micro-wells formed in the second substrate of FIG. 7D.

FIG. 7F shows metal deposited in and over the micro-wells formed in the second substrate of FIG. 7E.

FIG. 7G shows a replication molding tool for a cell culture apparatus.

FIG. 7H shows a modified substrate for use in forming micro-channels between micro-wells and micro-well arrays of a cell culture substrate.

FIG. 8A is a light microscopic image of HepG2C3A cells cultured on steep-sided/flat-bottomed micro-wells.

FIG. 8B is a fluorescence microscopic image of HepG2C3A cells cultured on steep-sided/flat-bottomed micro-wells.

FIG. 8C is a light microscopic image of HepG2C3A cells cultured on curved micro-wells.

FIG. 8D is a fluorescence microscopic image of HepG2C3A cells cultured on curved micro-wells.

### DETAILED DESCRIPTION

The invention will now be described in detail with reference to a few preferred embodiments, as illustrated in the accompanying drawings. In describing the preferred embodiments, numerous specific details are set forth in order to provide a thorough understanding of the invention. However, it will be apparent to one skilled in the art that the invention may be practiced without some or all of these specific details. In other instances, well-known features and/or process steps have not been described in detail so as not to unnecessarily obscure the invention. In addition, like or identical reference numerals are used to identify common or similar elements.

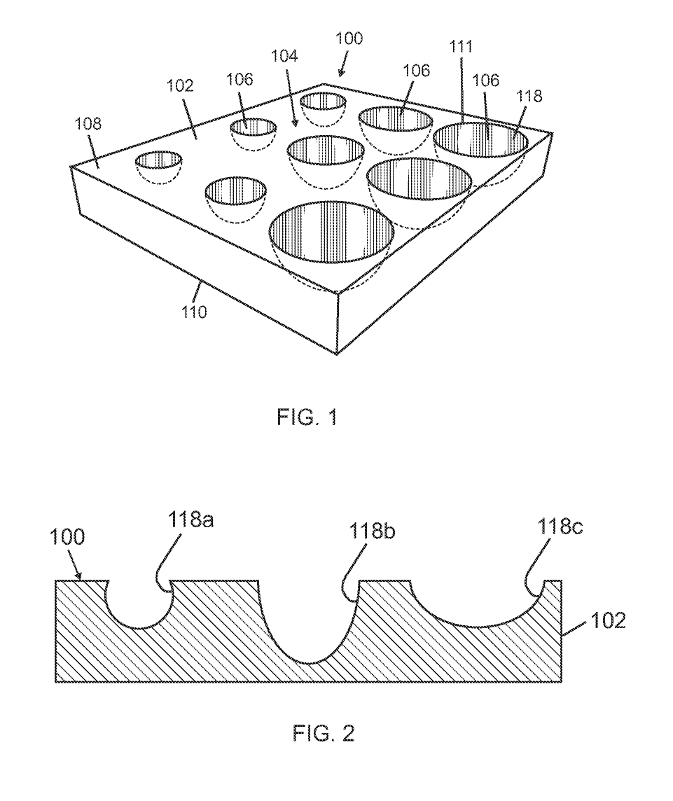

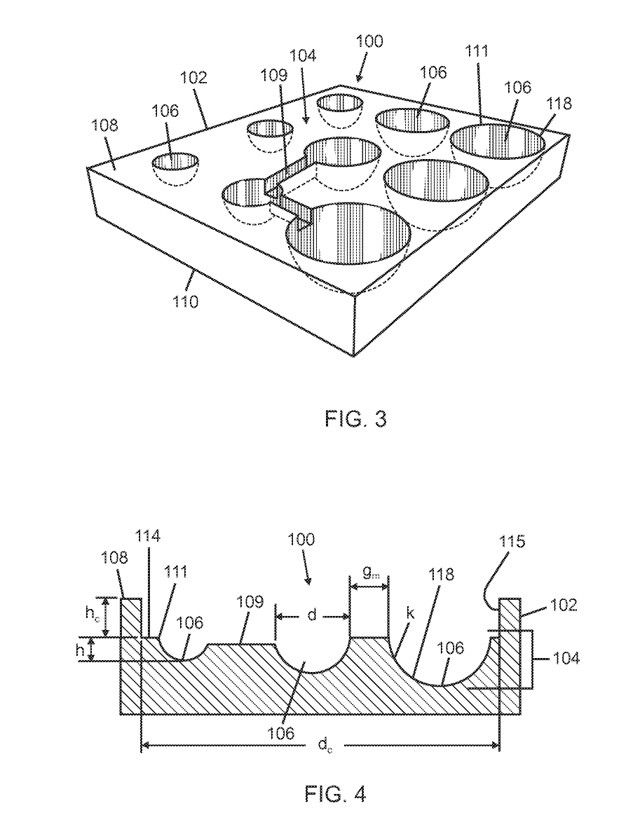

FIG. 1 depicts a cell culture apparatus 100 including a substrate 102, which may be made any biocompatible material suitable for cultivating biological cells. For example, the biocompatible material could be a biocompatible polymer, such as polydimethylsiloxane (PDMS), available commercially as silicone, poly(lactic-co-glycolic acid), polydimethylsiloxane, polyethylene, polystyrene, polyolefin, polyolefin copolymers, polycarbonate, ethylene vinyl acetate, polypropylene, polysulfone, polytetrafluoroethylene (PTFE) or compatible fluoropolymer, a silicone rubber or copolymer, poly(styrene-butadiene-styrene), acrylic, or polyester or combinations of these materials. Other examples of suitable materials include quartz, silicon dioxide, and titanium-coated silicon. A micro-well array 104 is formed in the substrate 102. The micro-well array 104 includes a plurality of micro-wells 106 formed in the substrate 102. The micro-wells 106 are spaced apart within the substrate 102. The spacing between adjacent micro-wells 106 may be uniform or non-uniform across the substrate 102. Each micro-well is defined by a curved surface 118 which is concave. In one embodiment, the curved surface 118 is an elliptic paraboloid. The term “elliptic paraboloid” also includes the special case of a circular paraboloid. In another embodiment, the curved surface is a segment of a spheroid, as illustrated in FIG. 2 at 118a. In yet another embodiment, the curved surface is a segment of an ellipsoid, as illustrated in FIG. 2 at 118b and 118c. The curved surface 118 which is concave is effective in confining three-dimensional cell cluster formation and enabling cell cluster growth in a true three-dimensional space.

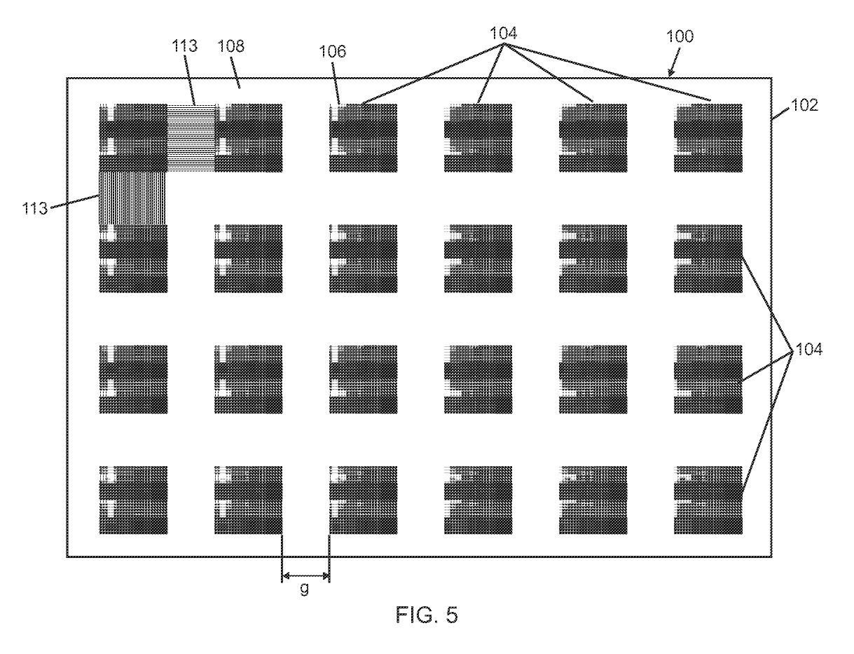

FIG. 3 shows that one or more pairs of micro-wells 106 in the cell culture apparatus 100 may be interconnected by micro-channels 109 formed in the substrate 102. It is not necessary that the interconnected micro-wells 106 are adjacent to each other. The micro-channels 109 may be suitably shaped to connect any desired pair of micro-wells 106. The micro-channels 109 enable the cells located within the bottom of micro-wells 106 to have free access to the cell culture medium, e.g., growth factors or stimulus, in the cell culture apparatus 100. The micro-channels 109 also promotes communication between the interconnected micro-wells 106. All or a portion of the micro-wells 106 in the cell culture apparatus 100 may be interconnected by the micro-channels 109. Each micro-channel 109 typically has a width that is smaller than the size of a single cell to be cultured in the micro-wells 106 that it interconnects. On the other hand, the micro-channel 109 could be larger than the size of the single cell to be cultured. In a preferred embodiment, the width of each micro-channel 109 ranges from 5 to 50 microns. The depth of the micro-channels 109 can be flexible but would generally not be larger than the depth of the shallowest micro-wells 106 to which it is connected.

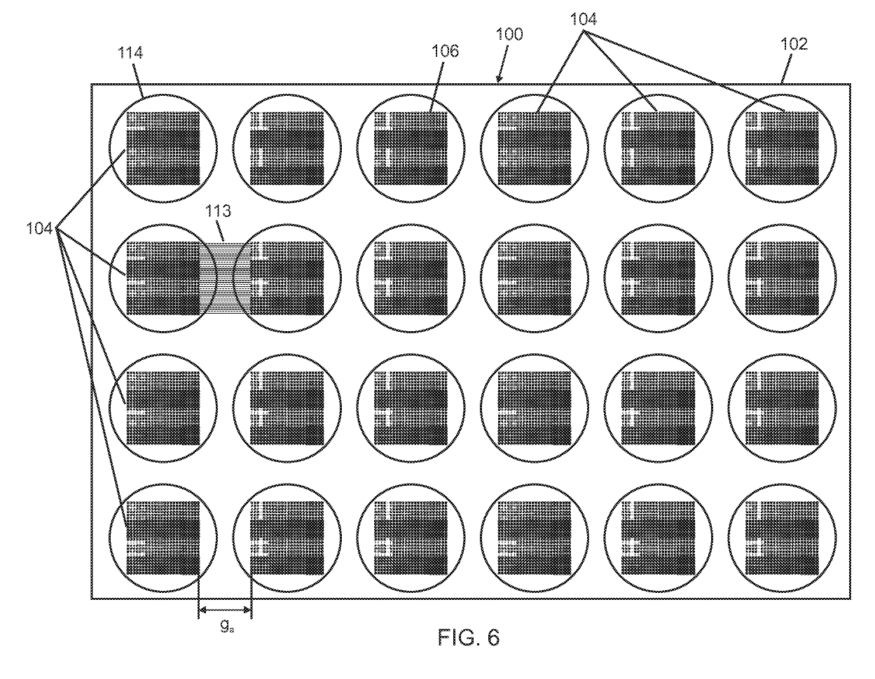

In FIGS. 1 and 3, the micro-wells 106 extend from the top surface 108 of the substrate 102 to a non-basal point (meaning that the micro-wells do not extend all the way through the substrate) in the substrate 102. The micro-wells 106 can have different depths and can terminate at different non-basal points within the substrate 102. The micro-wells 106 are not orifices in that they do not extend through to the bottom surface 110 of the substrate 102. In FIGS. 1 and 3, the rims 111 of the micro-wells 106 are flush with the top surface 108 of the substrate 102. FIG. 4 shows an alternate embodiment where the micro-well array 104 is formed at the bottom of a surface well 114 in the substrate 102 and the rims 111 of the micro-wells 106 in the micro-well array 104 are recessed relative to the top surface 108 of the substrate 102. The depth (h_{c}) of the surface well 114 determines how far the micro-well array 104 (or rims 111 of the micro-wells 106) is recessed relative to the top surface 108 of the substrate 102. The surface well 114 has a side wall 115, which in FIG. 4 is perpendicular to the top surface 108 of the substrate 102. In alternate embodiments, the surface well 114 may be slanted relative to the top surface 108 of the substrate 102. The surface well 114 may have various shapes, such as a cylindrical shape, an inverted, truncated conical shape, or a parallelepiped shape, with our without rounded corners. In the same manner as described above, micro-channel(s) 109 may be formed between the recessed micro-wells 106.

Referring again to FIG. 4, each micro-well 106 has a topography which is described by the following micro-well parameters: the diameter (d) of the micro-well 106, the height (h) of the micro-well 106, the curvature (k) of the micro-well 106, and any surface treatment (such as specialized coatings and/or textures) on the curved surface 118 of the micro-well 106. In a preferred embodiment, the diameter (d) of the micro-wells 106 ranges from 10 to 500 microns. In a preferred embodiment, the depth (d) of the micro-wells 106 ranges from 5 to 100 microns. The curvature (k) of the micro-wells 106 is bounded by the diameter and depth of the micro-wells 106. The micro-well array 104 has a topography that is described by the individual topographies of the micro-wells 106 within the array and the following micro-well array parameters: spacing (gₘ) between adjacent micro-wells 106, depth (h_{c}) of the surface well 114 if present, diameter (d_{c}) of the surface well 114 if present, shape of the surface well 114 if present, arrangement of the micro-wells 106 within the array, and the width and depth of micro-channels 109 if present. In a preferred embodiment, the spacing (gₘ) between adjacent micro-wells 106 ranges from 5 to 400 microns.

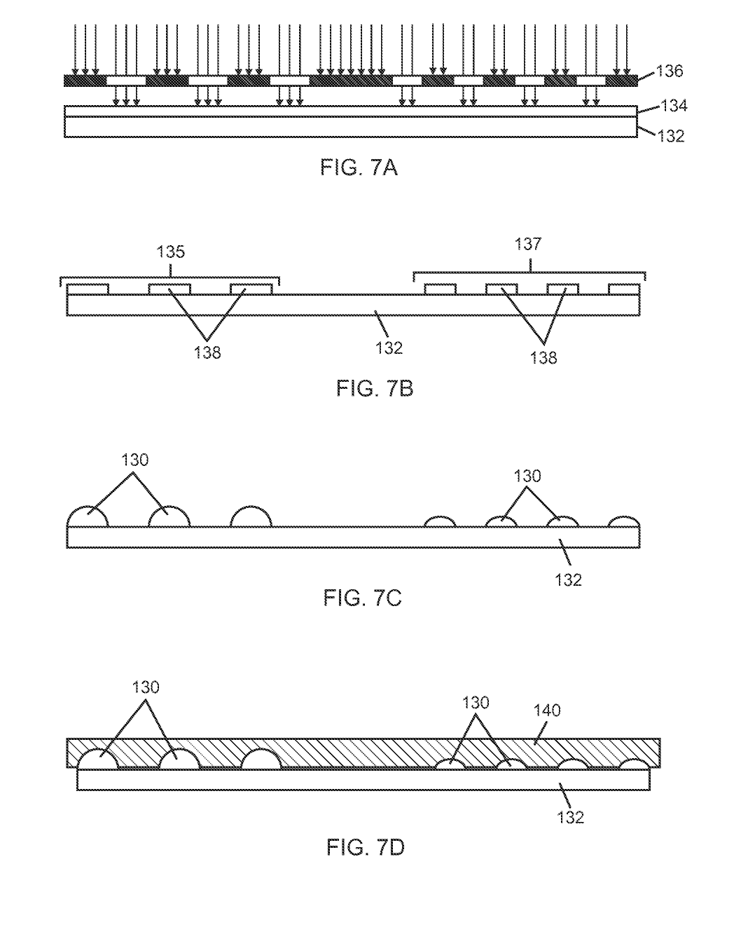

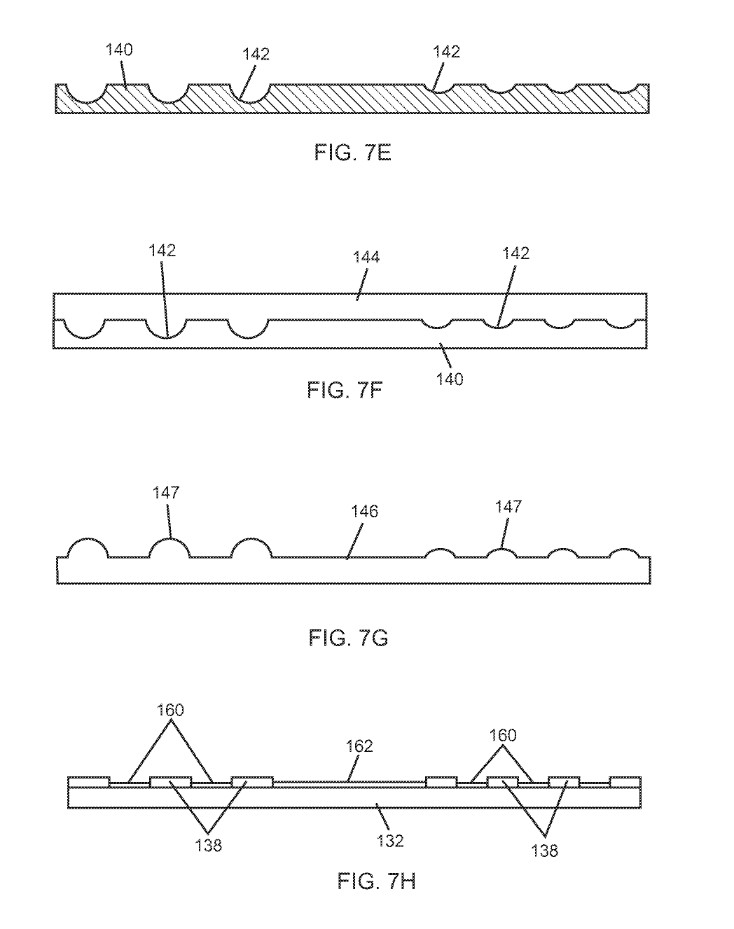

FIGS. 1 and 3 show the cell culture apparatus 100 as having a single micro-well array 104 with nine micro-wells 106. In practice, the cell culture apparatus 100 can have several more micro-wells 106 arranged in (or separated into) a plurality of micro-well arrays 104. In FIG. 5, for example, the cell culture apparatus 100 includes a plurality of micro-well arrays 104, where each micro-well array 104 includes a plurality of micro-wells 106. Because of the scale of the drawing, the micro-wells 106 appear as dots in this figure. In general, the micro-well array 104 would resemble the one shown in FIG. 1 or 3, except that in FIG. 5 there may be more micro-wells 106 per array and these micro-wells may have different topographies from that shown in FIG. 1 or 3. In FIG. 5, the micro-well arrays 104 may have equal number or different numbers of micro-wells 106. In FIG. 6, each micro-well array 104 (i.e., the general outline of the array) has a quadrate shape. In alternate embodiments, the micro-wells 106 may be arranged such that the micro-well array 104 has a non-quadrate shape, such as a circular or hexagonal shape. FIG. 6 shows the multi-arrayed cell culture apparatus with the micro-well arrays 104 formed at the bottom of surface wells 114, as previously described with respect to FIG. 4.

In the same manner that micro-channels (109 in FIGS. 3 and 4) can be formed between pairs of micro-wells 106 in a micro-well array 104, micro-channels can also be formed between pairs of micro-well arrays 104 in a cell culture apparatus 100. This is suggested by lines 113 in FIG. 5. The lines 113 are actually micro-channels connecting micro-wells 106 in a first micro-well array 104 to micro-wells 106 in a second micro-well array 104. As many micro-wells 106 as desired may be interconnected by the micro-channels 113. Micro-channels 113 may also be used where micro-well arrays 104 are formed at the bottom of surface wells (114 in FIG. 6). In this case, the micro-channels 113 will cut through the surface wells 114 in other arrays to provide the desired interconnections. In FIGS. 5 and 6, the width of each micro-channel 113 may be smaller than the size of a single cell to be cultured in the micro-wells 106 to allow fluid to flow between arrays but to prevent cells from flowing between arrays. On the other hand, the micro-channel 113 may be larger than the size of the single cell to be cultured. In an embodiment, the width of each micro-channel 113 ranges from 5 to 50 microns. The depth of the micro-channels 113 can be flexible. The micro-channels 113 can promote communication between the micro-well arrays 104 it interconnects as well as enable the cells located within the bottom of the interconnected micro-well arrays 104 to have free access to the cell culture medium, e.g., growth factors or stimulus, in the cell culture apparatus 100.

Referring again to FIGS. 5 and 6, the micro-well arrays 104 are spaced apart within the substrate 102. The spacing (gₐ) between the micro-well arrays 104 may be uniform or non-uniform across the substrate 102. Any suitable arrangement of the micro-well arrays 104 in the substrate 102 may be used, but it may be convenient to arrange the micro-well arrays 104 as an N×M rectangular or square array, where N>1, M≧1. In one example, the cell culture apparatus 100 is in a micro-titer plate format with N×M having a value selected from 2, 4, 8, 24, 96, 384, and 1536.

Referring to FIGS. 1-6, the cell culture apparatus 100 may be configured as a screening tool for identifying optimum micro-environments for culturing of specific biological cells. Where the cell culture apparatus 100 is configured as a screening tool, at least two, preferably a more substantial number, of the micro-wells 106 have different topographies. Preferably, the different topographies of the micro-wells 106 are spread over domain of micro-well topographies to be investigated. Two micro-wells 106 have different topographies if at least one of the aforementioned micro-well parameters are different in value. The at least two micro-wells 106 having different topographies may be located within a single micro-well array 104 or in two different micro-well arrays 104. When the cell culture apparatus 100 is configured for a specific cell line, the micro-wells 106 in the cell culture apparatus 100 may have the same topography. For co-culturing of a plurality of cell lines, the cell culture apparatus 100 may include a plurality of micro-well arrays 104, wherein each micro-well array 104 includes micro-wells 106 tailored to a specific cell line. In this case, the micro-wells 106 within each micro-well array 104 may also have the same topography or whatever topographies have been determined to be best suited for the specific cell line. Earlier on, it was mentioned that each micro-well 106 has a topography and that each micro-well array 104 has a topography. The cell culture apparatus 100 also has a topography which is described by the collective topographies of the micro-well arrays 104 and the following cell culture apparatus parameters: arrangement and shape of the micro-well arrays 104 within the substrate and arrangement and dimensions of interconnecting micro-channels 113 between the micro-well arrays 104. Where the cell culture apparatus 100 includes several micro-wells 106 having different topographies, where the different topographies are spread over a domain of micro-well topographies to be interrogated, the cell culture apparatus 100 is said to have a range of different topographies.

The cell culture apparatus 100 described above in FIGS. 1-6 can be used to cultivate three-dimensional multicellular clusters of cells. The micro-wells 106 can mimic the essential environment of in-vivo cells and facilitate nutrition and metabolic waste transportation. As a screening tool, the cell culture apparatus 100 can facilitate discovery of optimum or effective microenvironment for three-dimensional cell culture of different types in that the influence of microenvironment on cell culture can be rapidly and effectively studied by comparing a broad range of cell culture results on a single multi-well plate or chip. In particular, the cell culture apparatus 100 makes it easy to investigate the effect of changes in micro-well parameters on the cell culture microenvironment on a single chip. The location of each micro-well 106 in the substrate and the arrangement of the micro-wells 106 in the substrate 102 are known. This information can be stored in any suitable format for later use in identifying the micro-wells 106 (or micro-well arrays 104) that behave optimally or effectively in terms of cell-substrate interaction for a particular cell type. In one example, the location and description of the micro-wells 106 are stored in a structured file, such as an XML file, or other computer-readable structured format. The stored location and description of the micro-wells 106 are then used to create an initial digital image of the cell culture apparatus 100. After initiation of a cell-substrate interaction study, subsequent digital images of the cell culture apparatus 100 are generated. These subsequent digital images may be compared to the initial image to assist in identifying micro-wells 106 (or micro-well arrays 104) that behave optimally.

These steps may be discussed as a four-step process where an initial scan is performed in step 1 and initial scan data is stored related to the micro-well array and its parameters. In step 2, cells are grown in the micro-well array(s). In step 3, a second scan is performed to measure cell growth and cell phenotype information. In step 4, the second scan data obtained in step 3 is compared to the initial scan data obtained in step 1, and this data is analyzed to identify the wells in the micro-well array which provided optimal cell growth and phenotype characteristics.

For completeness, an example of a method of making the cell culture apparatus described above is presented herein. However, the cell culture apparatus described above could be made via any suitable process or combination of processes known in the art.

In one example, a method of making the cell culture apparatus as described above includes forming one or more micro-dot arrays on a first substrate. Each micro-dot array includes a plurality of micro-dots, where each micro-dot is a negative copy of a micro-well as described above. The micro-dots may be formed on the first substrate by any suitable process such as casting or photolithography, followed by resist reflow. The micro-dot array(s) is(are) impressed into a second substrate to form micro-well array(s) in the second substrate. The process may include further steps where the cell culture apparatus includes a surface well above the micro-well array and/or interconnect channels between micro-wells.

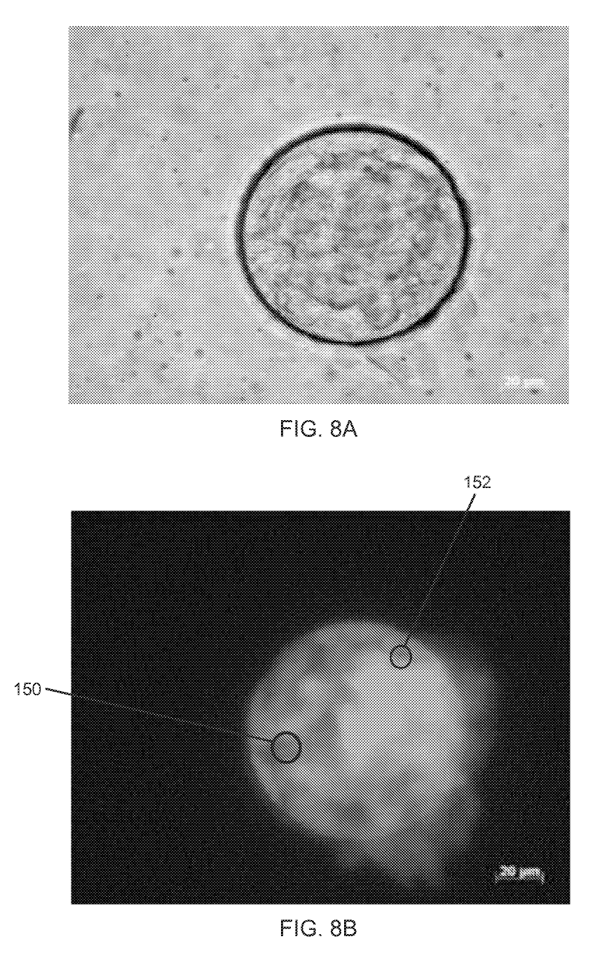

FIGS. 7A-7B illustrate a method of forming one or more micro-dot arrays on a substrate. In FIG. 7A, a photoresist 134 is deposited on a first substrate 132 using any suitable process known in the art, such as spin coating. The first substrate 132 may be made of any suitable substrate material, such as glass, polymer, or silicon. Preferably, the first substrate 132 has a hydrophilic surface to facilitate adhesion to the photoresist 134. Preferably, the photoresist 134 is a positive photoresist. The thickness of the photoresist 134 on the first substrate 132 is generally determined by the final depth of the micro-wells to be formed. Next, the photoresist 134 is exposed to a pattern of light through a photomask 136. The pattern of light is determined by the configuration of a micro-well array to be formed. The exposed photoresist 134 is developed and etched to form micro-posts. FIG. 7B shows the micro-posts 138, arranged in two adjacent arrays 135, 137. Only a slice of the substrate 132 with the micro-posts 138 is shown to simply the illustration. Also, to avoid crowding the figure, only a few micro-posts 138 in the two adjacent arrays 135, 137 are shown.

The micro-posts 138 in FIG. 7B are shaped into micro-dots 130 in FIG. 7C by a resist reflow process. As previously mentioned, the micro-dots are negative copies of the micro-wells to be formed. The resist reflow process includes heating the micro-posts 138 in FIG. 7B above the glass transition temperature of the first substrate material, wherein the micro-posts 138 deform by surface tension to form the micro-dots 130 in FIG. 7C. The shape of the micro-dots 130 in FIG. 7C is governed by the height and diameter of the micro-posts 138 in FIG. 7B and controlled by the degree and duration of the heat applied to the micro-posts.

Referring to FIG. 7D, a second substrate 140 is brought into contact with and pressed against the micro-dots 130 so that impressions of the micro-dots 130 are formed in the second substrate 140. These impressions become the micro-wells. Where the second substrate 140 is intended as the final product, the second substrate 140 is preferably made of a biocompatible material, as described with respect to the substrate of the cell culture apparatus above. FIG. 7E shows the second substrate 140 with the impressions 142. The process of making the cell culture apparatus may further include modifying the surfaces of the impressions (or micro-wells) 142. For example, such modification may include coating the surfaces of the impressions with a coating material such as collagen or other material desired to provide a specific micro-environment to be investigated. Another modification may be applying a texture to the surfaces of the impressions 142.

To facilitate large-scale production of the cell culture apparatus, it may be desirable to form a replication molding tool using the second substrate 140 with the impressions (micro-wells) 142. Referring to FIG. 7F, this may include, for example, depositing metal 144 in and over the impressions (micro-wells) 142 and over the top surface of the second substrate 140 by a process such as electroplating. The deposited metal may be separated from the second substrate 140 and subsequently used as the replication molding tool, which has the negative copies 147 of the desired micro-wells. FIG. 7G shows the replication molding tool 146.

Similarly, a cell culture apparatus having interconnected micro-channels can be fabricated using the above-mentioned standard approaches. For example, as illustrated in FIG. 7H, spacer posts 160, 162 can be formed between the micro-posts 138 on the first substrate 132 by modifying the photo-pattern applied in FIG. 7A. The micro-posts 138 can be reshaped into micro-dots, as described above. The spacer posts 160, 162 will not be reshaped and will intersect with the micro-dots. The micro-dots and spacer posts can then be impressed into a second substrate, as demonstrated in FIG. 7D, to form micro-wells and micro-channels, respectively, in the second substrate, where the micro-channels would interconnect the micro-wells. The spacer posts 160 would result in micro-channels within the micro-well arrays, while the spacer post(s) 162 will result in micro-channels between the micro-well arrays.

A cell culture apparatus in which micro-well arrays are formed at the bottoms of surface wells can be formed using, for example, a photolithography process, such as described above, or any other suitable process for forming an array of trenches in a substrate, where the array of trenches would serve as the array of surface wells. Then, a replication molding tool formed as described above can be used to stamp the bottom of the surface wells with the micro-well arrays. Alternatively, an array of macro-posts separated by trenches could be formed on a first substrate, where the macro-posts would serve as negative copies of the surface wells. Then, micro-posts could be formed on the macro-posts. The micro-posts could be shaped into micro-dots, which would serve as negative copies of the micro-wells, as described above. Then the macro-posts and micro-dots can be impressed into a second substrate to form the cell culture apparatus. Alternatively, a substrate including orifices, which would serve as the surface wells, may be formed and adjoined to a substrate including micro-wells. Any suitable process may be used to adjoin the substrates.

HepG2C3A is a tumor liver cell line. The liver is the primary site of detoxification of many toxic substances from the blood, as well as the synthesis and secretion of many compounds. Hepatocytes, the most abundant cells that make up 70-80% of the cytoplasmic mass of the liver, carry out most of the metabolic and biosynthetic processes in liver. Thus, in vitro cultured hepatocytes are popular for drug metabolism and toxicity studies. These studies are traditionally based on two-dimensional cell cultures. However, many cells, including hepatocytes, lose their polarity in culture. As a result, primary hepatocytes in traditional two-dimensional culture rapidly lose their ability for drug metabolism and transporter functions.

Morphology and viability of hepatocyte HepG2C2A cells cultured onto two different types of micro-wells—prior-art micro-wells having steep side wall and flat bottom and micro-wells having curved surfaces as described with respect to FIGS. 1-6—were studied. For the study, micro-well arrays having the different types of micro-wells were formed in poly-dimethylsiloxane (PDMS) substrates. The final PDMS substrates having the micro-wells were formed by curing the PDMS pre-polymer solution containing a mixture of PDMS oligomers and a reticular agent from Sylgard 184 kit in a 10:1 mass ratio on a silicon master. The PDMS was thermally cured at 70° C. for 80 minutes. PDMS molds with very high fidelity to a patterned template. Once the PDMS substrates were made, these substrates were subjected to surface oxidation using O₂ plasma cleaning for 30 seconds at a pressure of 500 mTorr. The micro-well arrays were arranged at the bottom of (surface) wells in a 24-well microplate. Afterwards, each well was filled with 75% ethanol twice, each fill lasting 30 seconds, followed by washing with PBS (phosphate saline) buffer and drying. Finally, a PBS-buffered Collagen I solution (200 μL) was added into each well, and the wells were incubated for 45 minutes. After aspiration of the Collagen I solution, the surface of each well was air-dried. HepG2C3A (CRL-1074) human hepatoblastoma cell line from American Type Culture Collection was cultured in MEM Eagle medium containing 1 mM sodium pyruvate, 10% (v/v) fetal bovine serum (FBS), and 2 mM L-glutamine. All cell cultivation, HepG2C3A cells was seeded in the 24-well plates. The cells were cultured under standard conditions: a humidified atmosphere of 5% CO₂ and 95% air at 37° C. with daily medium changes. A duplicate for each conditions was examined. The Live/Dead staining was carried out using the protocol recommended by the manufacturer. All microscopic images were obtained using Zeiss microscope.

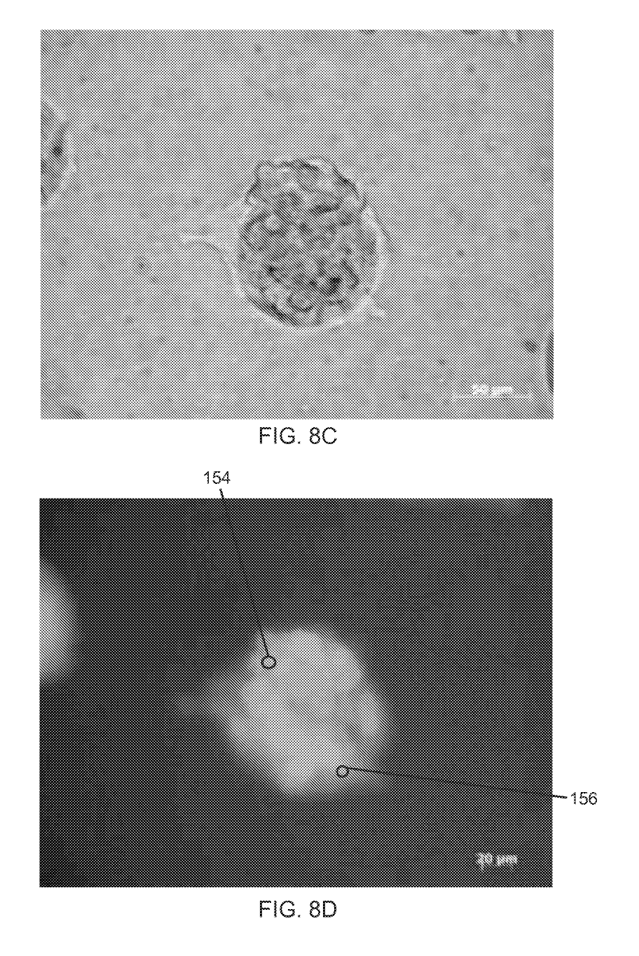

FIG. 8A and FIG. 8B show light and fluorescence microscopic images, respectively, of HepG2C3A cells cultured onto the steep-sided/flat-bottomed micro-wells. The fluorescence image was obtained after five day culturing under normal culture condition, followed by LIVE/DEAD staining with the LIVE/DEAD staining reagent kit from Molecular Probes Inc (Eugene, Oreg.). Green color indicated the cells were alive, while red color indicated the cells were dead. The dead cells are within the circled regions 150, 152 in FIG. 8B. Both images showed that the majority of cells were still alive, although some cells closer to the bottom of the micro-well were dead. The cells exhibited largely 2-dimensional morphology. That is, the cells were flat and mostly well spread.

FIG. 8C and FIG.8D show light and fluorescence microscopic images, respectively, of HepG2C3A cells cultured onto the curved micro-wells. The fluorescence image was obtained after five days of culturing under normal culture condition, followed by LIVE/DEAD staining with the LIVE/DEAD staining reagent kit from Molecular Probes Inc (Eugene, Oreg.). Both images showed that almost all cells are still alive. Regions with dead cells are indicated at 154, 156 in FIG. 8B. The cells exhibited largely 3-dimensional morphology. That is, the cells were round with no obvious spreading. The cells also had a spheroid shape.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A cell culture apparatus, comprising:
a substrate having a plurality of micro-well arrays;
wherein each micro-well array has a plurality of micro-wells;
wherein at least two of the micro-wells have different topography.

2. The cell culture apparatus of claim 1 wherein each microwell is curved.

3. The cell culture apparatus of claim 1, wherein each of the curved micro-wells has a concave shape.

4. The cell culture apparatus of claim 2, wherein at least one of the micro-well arrays comprises a heterogeneous set of curved micro-wells.

5. The cell culture apparatus of claim 2, wherein at least one of the micro-well arrays comprises a homogeneous set of curved micro-wells.

6. The cell culture apparatus of claim 1, wherein the micro-well arrays are located adjacent to a first boundary surface of the substrate and are accessible through the first boundary surface of the substrate.

7. The cell culture apparatus of claim 1, a total number of the micro-well arrays in the substrate is selected from the group consisting of 6, 12, 24, 96, and 384.

8. The cell culture apparatus of claim 2, wherein each of the curved micro-wells has a diameter in a range from 10 to 400 microns.

9. The cell culture apparatus of claim 2, wherein each of the curved micro-wells has a depth in a range from 5 to 50 microns.

10. The cell culture apparatus of claim 1, further comprising a plurality of macro-wells formed in the substrate, each macro-well containing at least one micro-well array.

11. The cell culture apparatus of claim 10, wherein the micro-well arrays are accessible through the macro-wells.

12. The cell culture apparatus of claim 10, wherein each of the curved micro-wells has a diameter in a range from 10 to 400 microns.

13. The cell culture apparatus of claim 10, wherein each of the curved micro-wells has a depth in a range from 5 to 50 microns.

14. A method of making a cell culture apparatus, comprising:
forming a plurality of microdots on a first substrate, at least two of the microdots having different topographies, each microdot being a negative copy of a curved micro-well;
impressing the microdots into a second substrate to form a plurality of micro-wells in the second substrate.

15. The method of claim 14, wherein forming the plurality of microdots comprises depositing a photoresist on the first substrate, exposing the photoresist to a pattern of light, and developing and etching the exposed photoresist to form a plurality of micro-posts on the first substrate.

16. The method of claim 14, further comprising shaping the micro-posts into microdots by resist reflow.

17. A method of using the cell culture apparatus of claim 1 comprising the steps of:
(1) obtaining data about the cell culture apparatus;
(2) culturing cells in the micro-wells of the cell culture apparatus;
(3) obtaining data about the cells cultured in the micro-wells of the cell culture apparatus; and,
(4) comparing the data from step (1) with the data from step (3) to determine optimal micro-well parameters for cell culture.
